(19) United States
(12) Patent Application Publication
(10) Pub. No.: US 2023/0172462 A1
(43) Pub. Date: Jun. 8, 2023

(54) Device And Method For Sleeve Shield and Wrap Shield For Hygienic Protection

(71) Applicant: Brenda Perkins Sweat, Fayetteville, GA (US)

(72) Inventor: Brenda Perkins Sweat, Fayetteville, GA (US)

(21) Appl. No.: 17/545,199

(22) Filed: Dec. 8, 2021

(51) Int. Cl. A61B 5/022; A61B 46/10

(52) U.S. Cl. CPC A61B 5/02233; A61B 46/10; A61B 2562/247

(57) ABSTRACT

The disclosed inventive concept is essentially a hygienic shield. The hygienic shield may comprise two basic embodiments, referred to as a Cuff Sleeve Shield and a Cuff Wrap Shield, respectively. Both versions of the hygienic shield are disposable immediately after use. The preferred embodiment and method of use of the hygienic shield is as a protective blood pressure (BP) cuff shield, constructed as a tubular apparatus made of thin, highly flexible rubber. In the alternate embodiment and method of use, the hygienic shield is fabricated from a planar segment of rectangular material, preferably blue nitrile rubber of 4.0 mm thickness. The Cuff Wrap Shield further is fabricated with a first means of attachment at a first side of the Cuff Wrap Shield and a second means of corresponding attachment integral to a directly opposing second side. Thus, when joined at the seam, there is ultimately formed a tubular sleeve apparatus.

### CROSS-REFERENCES TO RELATED APPLICATIONS

This patent application claims the benefit of priority from previously-filed U.S. provisional patent application, Ser. No. 63/090,166, filed on Oct. 9, 2020 as though fully appearing herein. This patent application further claims the benefit and priority of a Petition, pursuant to 37 CFR 1.78(c), filed with the Commissioner of Patents on Dec. 8, 2021, said Petition seeking an extension of time of two (2) months for the effectivity of the filing date of provisional patent application No. 63/090,166.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### BACKGROUND OF THE INVENTION

### (1) Field of the Invention

The field of the present inventive concept relates generally to methods and equipment that may be utilized by medical personnel for providing a protective shield or wrap to avoid cross-contamination between a blood pressure cuff (or other medical device) and the skin of a human wearer when, the cuff or device is to be placed on the arm, leg or other body part of the user or wearer.

### (2) Description of the Related Art, Including Information Disclosed Under 37 CFR 1.97 and 1.98

U.S. Published Patent Application 2007/0142794 A1; Jun. 21, 2007, Bester, W.

A hygienic article for personal wear by a male includes an elongate fabric sleeve having a first end, a longitudinally opposite second end, and a central passage extending longitudinally between the first and second ends of the sleeve. The second end of the sleeve is also open to the central passage to permit the wearer to urinate through the open second end. A dispenser is adapted for holding a plurality of such hygienic articles and facilitating sequential dispensing of the hygienic articles, one at a time, from the dispenser.

U.S. Published Patent Application, 202/0095106 A1; Caliguire, P. Jul. 18, 2002;

A carpal tunnel support for use by a wearer in the operation of components located above a work surface consisting of a 12″ flexible sleeve preferably made from nylon, and having a strip of hook and loop material fastened along its length and an elongated support panel having a strip of hook and loop material fastened along its length, the support panel is attached to the sleeve with the hook and loop material.

U.S. Pat. No. 10,383,772; Brown, R. Aug. 20, 2019

The present invention relates to a skin protection device comprising: a flexible cover having a first end and a second end. The cover comprises two sides extending between said first and second ends. The sides comprise seal sections capable of mutual engagement to form a seal. Each seal section includes a surface that is longitudinally serrated or undulating, wherein serration or undulation is provided on both sides of the section, a first female serrated or undulating side and a second male serrated or undulating side which when placed together form a mating pair and the seal. Each seal section is an extrusion as one part with a flat base and a plurality of serrations or undulations, folded and secured around the sides of the cover so that the flat base is in contact with both surfaces of the cover.

U.S. Pat. No. 6,525,238; Corrales, E; Feb. 25, 2003;

A single use disposable skin and cuff protector in the form of a wrap for surrounding at least a portion of a limb of a patient allows a sphygmomanometer cuff to be applied over the wrap. To prevent spreading of pathogens, the wrap includes a sheet having a non-porous barrier layer and optionally, an absorbent layer. The wrap is secured by a non-reusable fastener which cannot be released once fastened or once fastened, cannot be re-fastened. A line of weakening formed in the sheet facilitates removal of the wrap after use. Once secured to a patient for use, the sheet either must be severed to be removed and/or the re-usable fastener cannot be re-fastened thus preventing re-use.

U.S. Pat. No. 10,349,841; Li, Guang; Jul. 16, 2019

A blood pressure cuff assembly for an ambulatory blood pressure monitor may include a hollow cuff and an elastic liner provided on the reverse side of the cuff. An airbag may be provided in the cuff. A first connection part and a second connection part may be provided on the cuff. A third connection part and a fourth connection part may be correspondingly provided on the liner. The effect of tight inside and loose outside is achieved by the combination of the elastic liner and the cuff. When worn on an upper arm, the elastic liner has an elastic deformation and consequently tightens around the upper arm so as to realize certain slip resistance, and the cuff will not slip down even tied a bit loosely when located on the upper arm. The feeling of constraint from the cuff is reduced significantly and the comfort is improved.

### BRIEF SUMMARY OF THE INVENTION

The disclosed inventive concept is, in essence, a hygienic shield 1. The hygienic shield 1 may be formed by two methods resulting in two basic embodiments, referred to as a Cuff Sleeve Shield 1, 10, and a Cuff Wrap Shield 1, 20, respectively. Both embodiments 10, 20 of the hygienic shield 1 are items intended to be disposable immediately after use.

The preferred embodiment and method of use of the hygienic shield 1 is as a disposable blood pressure (BP) Cuff Sleeve Shield 10, constructed as a flexible, tubular apparatus made of blue nitrile rubber of 4.0 mm thickness. In the secondary embodiment and method of use, the Cuff Wrap Shield 20 is fabricated from planar, rectangular material, also made of blue nitrile rubber of 4.0 mm thickness. The Cuff Wrap Shield 20 further is fabricated with at least one means of attachment of a first edge 23 of the Cuff Wrap Shield 20 to a directly opposing second edge 24, thereby forming a seam 27.

The Cuff Wrap Shield 20, in its initial state, exists as a planar section of material and, in the preferred embodiment, a rectangle 28. Upon coordinated attachment of a first edge 23 of the rectangle 28 to a directly opposite, parallel, second edge 24, a tubular sleeve apparatus, the Cuff Wrap Shield 20 is formed. The Cuff Wrap Shield 20 thus formed, exhibits identical dimensions as that of a similarly-sized, pre-formed Cuff Sleeve Shield 10. It is to be noted, however, other planar forms can be utilized, including a circle 35, 36 and a diamond-shaped quadrangle 37, 38.

### Objectives and Purposes of the Inventive Concept

A primary purpose of the inventive concept is to provide a hygienic shield 1 made of non-porous, non-allergenic rubber which is easy to use, disposable, with no cleaning requirement. Further, the hygienic shield 1 must protect human patients from cross-contamination of infections, diseases and germs in circumstances where a device or item of medical equipment may be placed against the skin of a human.

An important objective is where a blood pressure (BP) cuff is to be used for acquiring blood pressure or pulse readings from a human wearer, the hygienic shield 1, 10, 20 will be placed on the arm of the human wearer, whereby the BP cuff will not touch the skin when taking the necessary readings. Both embodiments of the hygienic shield 1 are made of nitrile rubber, 4.00 mm thick, which is the same medically approved PPE nitrile rubber that medical disposable gloves are made of.

It is an objective of this concept that the hygienic shield 1 may be fabricated from other than rectangular shapes, including a circle, rhombus, quadrilateral, hexagon, pentagon, octagon, triangle, or square. Further, each of the shapes will be made in other thicknesses from 2.0 mm to 12.0 mm. The hygienic shield 1 will also be made in a wide variety of sizes, ranging from use in providing protection to a newborn to a bariatric sized person from 1″×2″ to 1″×150.″ All hygienic shields 1 fabricated from a planar material will have a first attachment means 30 and a second attachment means 31 on respective opposite sides of the material.

In the use and functioning of the hygienic shield 1, an important objective is that the hygienic shield 1, 10, 20 (1) be non-porous, (2) be non-allergenic, (3) consist of rubber material that is thin, (4) consist of rubber that is as flexible as thin cloth material, (5) have a type of rubber that enables the determination of the location of a wearer's artery easier, (6) make it possible for a wearer's appropriate artery to be felt underneath the rubber, (7) enable visual sighting of the artery pulsating under the rubber, (8) is disposable, (9) is fixed at a very low cost to manufacture, and (10) requires a minimum of storage space.

A primary positive factor in the disclosed inventive concept is that it is simply designed, with a central tubular passage, easy to use, and very convenient for both medical practitioners and any person in the general public having need of such a device.

### BRIEF DESCRIPTION OF THE VIEWS OF DRAWINGS AS EXEMPLARY EMBODIMENTS OF THE INVENTIVE CONCEPT

FIG. 1 is a perspective view of a tubular sleeve apparatus, including its exterior surface 11, the interior surface 12, a first end 13, and a second 14. The apparatus is designated as the Cuff Sleeve Shield 10.

FIG. 2 presents a perspective view of a device referred to as a Cuff Wrap Shield 20, which, in its useable form, is of dimensions and function identical to the Cuff Sleeve Shield 10.

FIG. 3 illustrates the manner of bunching into gathers 18, the material at one end of the Cuff Sleeve Shield 10, by which a user is more readily enabled to place the Cuff Sleeve Shield 10 about some part of the anatomy of the user.

FIG. 4 is a direct view of the exterior surface 22 of the original material of a rectangle 28 shape, utilized in forming the Cuff Wrap Shield 20, further showing a segment of material which functions as a first attachment means 30, at the upper end 25 of the rectangle.

FIG. 5 is a direct view of the interior surface 21 of the original material of a rectangle 28 shape, needed to form the Cuff Wrap Shield 20, further showing a segment of material which functions as a second attachment means 31, at the lower end 26 of the rectangle 28.

FIG. 5A is a side view of the left edge of the Cuff Wrap Shield 20, further showing a first attachment means 30 and a second attachment means 31.

FIG. 6 displays the positioning of the exterior surface 22 of the rectangular-shaped 28 material in the process of being rolled over so as to join the first attachment means 30 with the second attachment means 31 on the interior surface 21 of the rectangle.

FIG. 7 shows the end result of the process of FIG. 6, resulting in the joining of the first attachment means 30 to the second attachment means 31, thereby forming the tubular Cuff Wrap Shield 20.

FIGS. 8 through 8C, display views of two different embodiments of planar shapes that may also be utilized in forming a different configuration of a Cuff Wrap Shield 10. The first embodiment is a round, or circular form, further showing its exterior surface 35 and its reverse, interior surface 36, while the second embodiment is in the form of a diamond, further showing an exterior surface 37, and an interior surface 38.

Table of Nomenclature & Part Numbers of Invention

| | |
|---|---|
| 1. | Hygienic Shield |
| 2.-9. | n/a |
| 10. | Cuff Sleeve Shield |
| 11. | Exterior surface |
| 12. | Interior surface |
| 13. | First end |
| 14. | Second end |
| 15. | Thickness |
| 16. | Length (L) |
| 17. | Diameter (D) |
| 18. | Gathers |
| 19. | n/a |
| 20. | Cuff Wrap Shield |
| 21. | Interior surface |
| 22. | Exterior surface |
| 23. | First edge |
| 24. | Second edge |
| 25. | Upper end |
| 26. | Lower end |
| 27. | Seam |
| 28. | Rectangle |
| 29. | Length |
| 30. | First attachment means (A) |
| 31. | Second attachment means (B) |
| 32.-34. | n/a |
| 35. | Round exterior |
| 36. | Round interior |
| 37. | Diamond exterior |
| 38. | Diamond interior |
| 39. | n/a |
| 40. | Diamond length |
| 41. | Round diameter |

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The objects, features, and advantages of the inventive concept presented in this application, referred to generically as a “hygienic shield” 1. The inventive concept is more readily understood when referring to the accompanying drawings. The drawings, totaling nine figures, show the basic components and functions of embodiments and/or methods of use. In the several figures, like reference numbers are used in each figure to correspond to the same component as may be depicted in other figures.

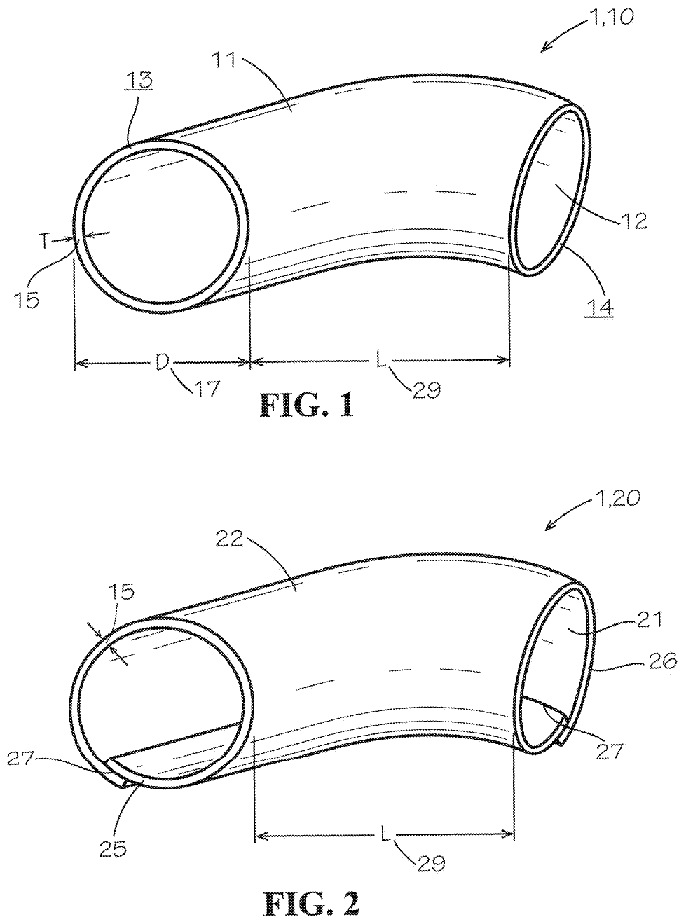

The discussion of the present inventive concept will be initiated with FIG. 1. In FIG. 1 there is illustrated a view of the preferred embodiment of the hygienic shield 1 in the form of a Cuff Sleeve Shield 10. FIG. 1 is a perspective view of an elongate rubber device, being a tubular sleeve apparatus. The view in FIG. 1 further shows its exterior surface 11, the interior surface 12, a first end 13, and a second end 14. The apparatus shown is designated as the Cuff Sleeve Shield 10.

In its preferred embodiment rendering, the Cuff Sleeve Shield 10 is composed of blue, nitrile rubber, of 4.0 mm thickness (T) 15. Thus, the Cuff Sleeve Shield 10 is inherently an extremely flexible, its interior surface forming a central tubular passage. The length and width dimensions of the Cuff Sleeve Shield 10 are variable, in accordance with the weight and anatomy structure of the intended wearer.

In its preferred embodiment, the Cuff Sleeve Shield 10 is intended for use as a hygienic shield 1 between the skin of a user (the wearer) and the surface of a blood pressure (BP) cuff. As commonly used, a BP cuff is applied at the upper arm, or bicep area of the anatomy of a wearer/user, thus measuring his/her blood pressure. A wearer of the Cuff Sleeve Shield 10 will first place the Cuff Sleeve Shield 10 about his/her upper arm area, with the exterior surface 11 of the Cuff Sleeve Shield 10 exposed. Immediately afterwards, the user of the Cuff Sleeve Shield 10, or a caregiver, will place a blood pressure cuff directly about the exposed length (L) 29 of the Cuff Sleeve Shield 10, thereby providing hygienic shielding of the user/wearer's skin from any type of contaminants or particles that may reside on the blood pressure cuff.

By way of further analysis, the optimum length (L) 29 of the Cuff Sleeve Shield 10 is 16.0 inches. Similarly, the optimum diameter (D) 17 of the Cuff Sleeve Shield 10 is approximately 4.7 inches, which accommodates the circumference of either bicep of the average person. The length 29 of the Cuff Sleeve Shield 10 is measured from the first end 13 of the Cuff Sleeve Shield 10 to the second end 14 of the device. The optimum diameter (D) 17 translates to a circumference, about the exterior surface 11 of the Cuff Sleeve Shield 10, of approximately 14.0 inches.

It is to be understood that both the length (L)29 and optimum diameter (D) 17 of the Cuff Sleeve Shield 10 may be altered and varied at the point of manufacture to provide this device in a size appropriate for use in accommodating the anatomy of infants as well as that of significantly obese individuals.

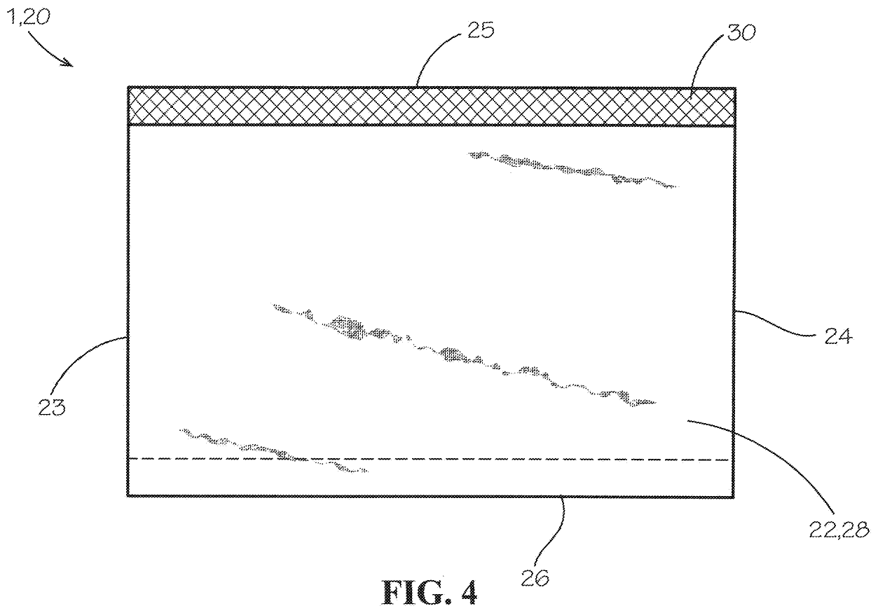

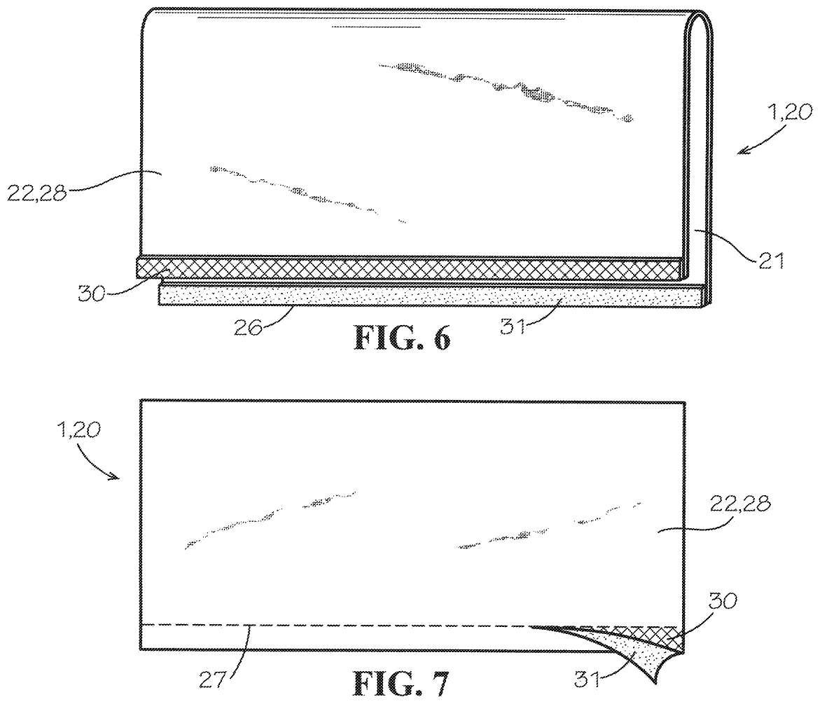

In FIG. 2, there is illustrated a view of an alternative embodiment of the hygienic shield 1 in the form of a Cuff Wrap Shield 20. As indicated in FIG. 2, the Cuff Wrap Shield 20 is a tubular-shaped sleeve apparatus, manifesting a seam 27. The seam 27 is formed by the sealed overlap of the upper end 25 and lower end 26 of a single piece of material, the material originally being a rectangle 28 shape. The upper end 25 and lower end 26 are more readily seen in FIG. 4 and in FIG. 6. In its preferred embodiment, the Cuff Wrap Shield 20 is composed of blue nitrile rubber, of 4.0 mm thickness (T) 15.

Continuing with FIG. 2, it is seen that the Cuff Wrap Shield 20 displays a partial view of the interior surface 21 of the original rectangle 28 of material. The exterior surface 22 is prominently displayed, both surfaces 21, 22 being components in forming the Cuff Wrap Shield 20. Both the interior surface 21 and exterior surface 22 are bounded by an upper end 25 and a lower end 26. These are the same bounding ends designated as components of the exterior surface 22.

In this manner, the Cuff Wrap Shield 20 in FIG. 2 functions as an inherently flexible, collapsible, tubular structure. In its preferred embodiment, the Cuff Wrap Shield 20 is intended for use as a hygienic shield 1 between the skin of a user and the interior surface of a blood pressure (BP) cuff. As commonly used, the BP cuff is applied on the skin of the bicep area of the anatomy of a human wearer, for the purpose of measuring his/her blood pressure and/or heart rate. Both the Cuff Sleeve Shield 10 and the Cuff Wrap Shield 20 must be of a length sufficient to make contact with the portion of the medical equipment which may impinge upon the skin of the human wearer;

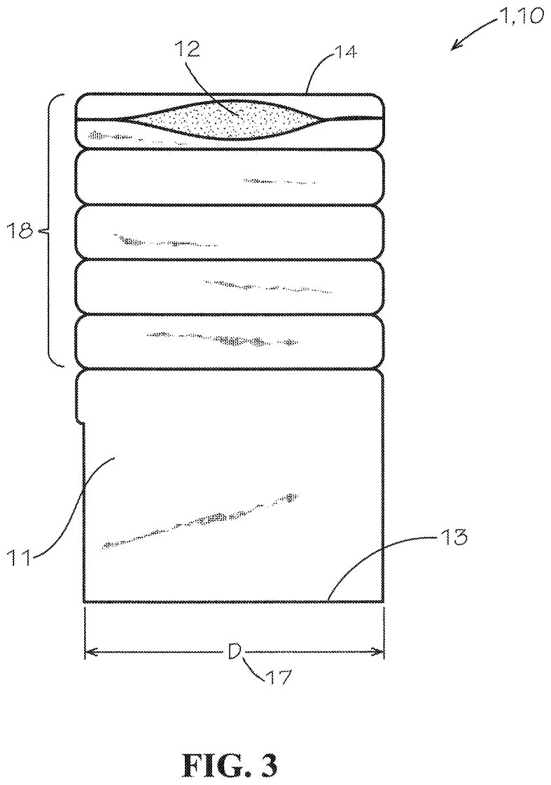

Directing attention to FIG. 3, there is displayed the initiation of a convenient process of placing the Cuff Sleeve Shield 10 about, for example, the forearm and bicep area of a wearer/user. FIG. 3 displays the manner of bunching into gathers 18, the material at the second end 14 of the Cuff Sleeve Shield 10. The gathers 18 thereby form the first portion of the Cuff Sleeve Shield 10 through which a user places his/her hand and wrist. The remaining material, extending to the first end 13 of the Cuff Sleeve Shield 10 will more easily follow in sequence. Likewise, in a similar manner, the process of displayed in FIG. 3 may, if necessary, also be used in donning the Cuff Wrap Shield 20.

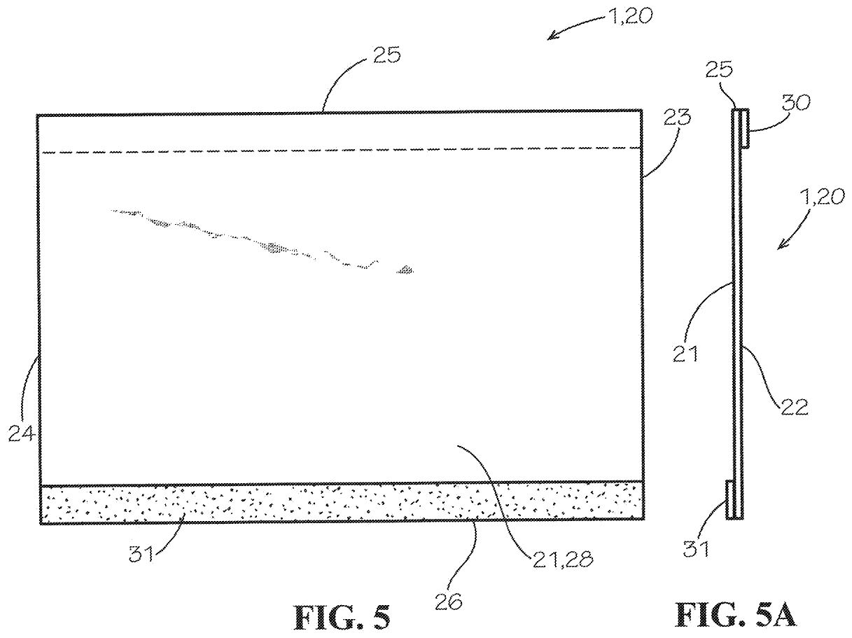

FIG. 4 presents a direct view of the exterior surface 22 of the original rectangle 28 of material, utilized in forming the Cuff Wrap Shield 20. Further shown, integral to the upper end 25 of the rectangle 28 material is a segment of material which comprises a first attachment means 30. The first attachment means 30 may be an adhesive, the felt component of a hook-and-felt fastening mechanism, a strip of covered adhesive tape, or other suitable attachment means. The exterior surface 22 shown is also bounded by a first edge 23, a second edge 24, an upper end 25, and a lower end 26. The reverse side of the view shown in FIG. 4 is the interior surface 21 of the rectangle 28 material, more clearly shown in FIG. 5.

FIG. 5, as mentioned previously, is a direct view of the interior surface 21 of the original rectangle 28 of material utilized in forming the Cuff Wrap Shield 20. The interior surface 21 is bounded by an upper end 25, a lower end 26, its first edge 23, and a second edge 24, all of which are also common to the exterior surface 22. Proximate the lower end 26 of the inter surface 21, there is displayed a second attachment means 31 which is fabricated so as to correspond to the first attachment means 30 integral to the exterior surface 22 of the rectangle 28. Similarly, the second attachment means 31 may be an adhesive, the hook component of a hook-and-felt fastening mechanism, a strip of covered adhesive tape, or other attachment means compatible with the first attachment means 30.

FIG. 5A is a side view of the first edge 23 of the Cuff Wrap Shield 20, further showing the placement of the first attachment means 30 onto the exterior surface 22 of the Cuff Wrap Shield 20, and the placement of the second attachment means 31 onto the interior surface 21 of the Cuff Wrap Shield 20. The tubular form of the Cuff Wrap Shield 20 is accomplished by a circular rolling of either the upper end 25 of the device or the lower end 26 so as to position the first attachment means 30 and second attachment means 31 against each other, forming a seam 27 (the seam 27 is also readily observed in FIG. 2 and FIG. 7). In this manner, a tubular sleeve apparatus is formed.

FIG. 6 displays the manner in which the exterior surface 22 of the rectangle 28 material is positioned so as to ultimately form the Cuff Wrap Shield 20. The rectangle 28 is shown having been “rolled over,” so as to expose its exterior surface 22 and simultaneously place the interior surface 21 in an internal configuration. This rolling over of the rectangle 28 material may be performed either totally out of physical contact with the prospective human wearer, or by placing, for instance, one of the biceps of a human wearer in abutment to the interior surface 21 of the rectangle 28 prior to beginning the roll-over procedure.

At this stage of the “roll over” procedure, it is seen that the first attachment means 30 and second attachment means 31 sufficiently proximate each other so as to form a common bond. The configuration of the rectangle 28 as shown in FIG. 6 shows a design which is easily useable by left-handed and/or right-handed people, thereby facilitating the sealing of the first attachment means 30 and the second attachment means 31, one to the other.

FIG. 7 displays the Cuff Wrap Shield 20 in its ultimate form as a tubular-shaped sleeve apparatus. There is further displayed a slight unpeeling of material, thus revealing the second attachment means 31 bonding to, and approaching a full bonded connection to the first attachment means 30.

FIG. 7 shows near completion of the “rollover” procedure of FIG. 6, resulting in the joining of the first attachment means 30 to the second attachment means 31. In this manner, there is formed a seam 27, evidencing the completion of the Cuff Wrap Shield 20. This same result would be obtained had, for instance, the upper arm area of a human wearer/user been placed flush against the interior of the rectangle 28 prior to initiating the rollover process.

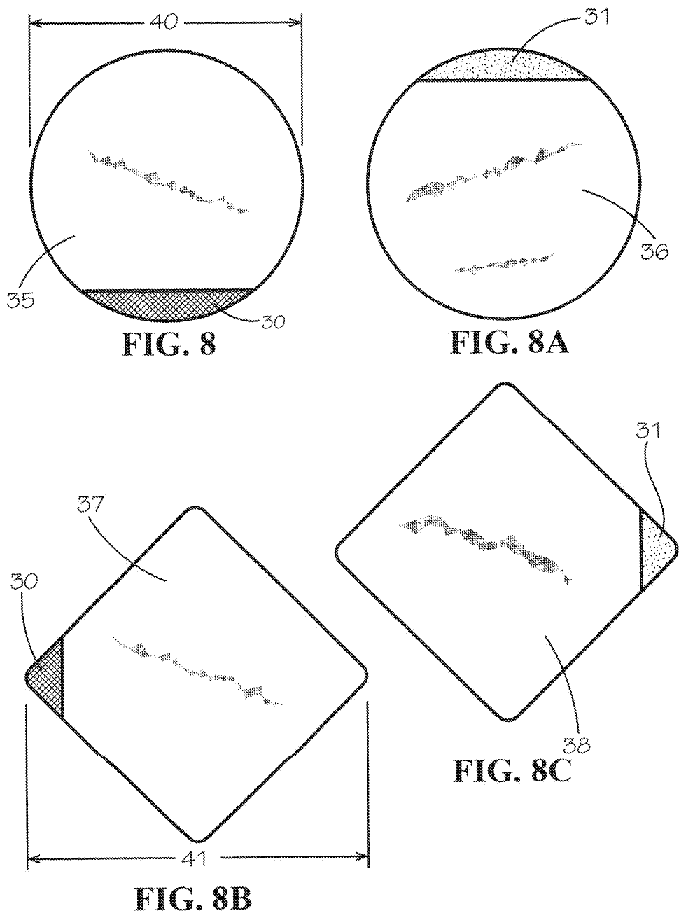

FIGS. 8, 8A, 8B, and 8C display views of two different embodiments of planar shapes that may also be utilized in forming a differently-shaped configuration of a Cuff Wrap Shield 10. The first embodiment is a round, or circular form, further showing, in FIG. 8 and FIG. 8A, its exterior surface 35 and its reverse, interior surface 36. The diameter 40 of the planar circle may be varied, prior to manufacture, to accommodate varying sizes of human anatomy for which use of these devices may be necessary.

In viewing FIG. 8B and FIG. 8C, there are shown views of a segment of material constructed in the form of a diamond, further showing an exterior surface 37, and an interior surface 38. Each of the views of the diamond-shaped planar material in FIGS. 8B and 8C also display a first and second means of attachment 30, 31. It is to be noted that the length of the diagonal 41 of the planar diamond shape may be varied, prior to manufacture, to accommodate the differing sizes of human anatomy for which use of this devices may be necessary.

The disposable blue nitrile rubber used in both the Cuff Sleeve Shield 10 and the Cuff Wrap Shield 20 is the same medically approved material that is utilized for personal protective equipment (PPE) in health care facilities. Just as a substantial quantity of rubber gloves are made of nitrile rubber, non-allergenic, non-porous and used as a PPE, similarly the Cuff Sleeve Shield 10 and Cuff Wrap Shield 20 will provide personal protection to the patient when placed on the arm or leg prior to many medical or health monitoring procedures.

While preferred embodiments of the present inventive method have been shown and disclosed herein, it will be obvious to those persons skilled in the art that such embodiments are presented by way of example only, and not as a limitation to the scope of the inventive concept. Numerous variations, changes, and substitutions may occur or be suggested to those skilled in the art without departing from the intent, scope, and totality of this inventive concept. Such variations, changes, and substitutions may involve other features which are already known per se and which may be used instead of, in combination with, or in addition to features already disclosed herein. Accordingly, it is intended that this inventive concept be inclusive of such variations, changes, and substitutions, as described by the scope of the claims presented herein.

What is claimed is:

1. A flexible, tubular sleeve apparatus for donning by a human wearer as hygienic protection of the skin of the wearer during the placement of a blood pressure cuff, any medical equipment or device around some part of the skin or anatomy of the wearer, the sleeve apparatus comprising an elongate rubber device (i) having an upper end which is parallel to a lower end, a first edge, a second edge, and a central tubular passage, (ii) manufactured from a rubber material; and (iii) of a length necessary to make contact with all portions of the medical equipment which may impinge upon the skin of the human wearer.

2. The tubular sleeve apparatus of claim 1, wherein the rubber material is blue nitrile rubber of 4.0 mm thickness, so as to enable (a) the determination of the location of the wearer's artery and (b) the wearer's appropriate artery to be felt underneath the rubber.

3. The tubular sleeve apparatus of claim 1, wherein (a) the medical equipment to be utilized comprises a blood pressure cuff, and (b) the skin of the anatomy is that of the upper arm area of a wearer.

4. The tubular sleeve apparatus of claim 1, wherein the sleeve apparatus is a continuous quantity of material fabricated in the form of an elongated, flexible cylinder.

5. The tubular sleeve apparatus of claim 1, wherein the sleeve apparatus is fabricated from material in the shape of a planar rectangle, where the rubber material is rolled into a cylindrical shape, formed by the two parallel opposing ends of the planar rectangle being mutually connected at a common seam.

6. The tubular sleeve apparatus of claim 1, wherein the dimensions of length and diameter of the central tubular passage are fabricated so as to accommodate the anatomy, weight, and size of the human wearer for which the tubular sleeve apparatus will be utilized.

7. The tubular sleeve apparatus of claim 1, wherein the sleeve apparatus is fabricated from material in the shape of a planar circle having a first means of attachment on a first side and a second means of attachment on the second side, where, upon rolling the circle material into a cylinder form, the first and second means of attachment are joined together, thereby forming the tubular sleeve apparatus.

8. The tubular sleeve apparatus of claim 1, wherein the sleeve apparatus is fabricated from material in the shape of a planar diamond having a first means of attachment on a first side and a second means of attachment on the second side of the material, where, upon rolling the diamond shape into a cylinder, the first and second means of attachment are joined together, thereby forming the tubular sleeve apparatus.

9. A method for safeguarding a human wearer from pathogens or harmful organisms that may be conveyed to the surface of the skin of the human when a health care procedure requires placement of a blood pressure cuff, an item of medical equipment or any type of medical device around or upon the skin of a limb of the wearer, the method comprising the steps of:
a) providing a continuous, tubular shaped sleeve apparatus, the sleeve apparatus comprising an elongate rubber cylinder having (i) an upper end, a parallel lower end, an interior surface, and an exterior surface; (ii) a central tubular passage, (iii) a material comprised of a type of thin rubber; (iv) a diameter approximately equivalent to the diameter of the subject limb of the human wearer, and (v) a length sufficient to make contact with all portions of the medical equipment which may impinge upon the skin of the human wearer;
b) placing the sleeve apparatus in direct perimetral contact with the limb about, or upon which the medical equipment is to be placed and ensuring that the interior surface of the sleeve apparatus is (i) placed flush against the skin of the wearer, so as to be absent any folds or gathers;
c) placing the item of medical equipment about, or in abutment with, the exterior surface of the sleeve apparatus; and
d) at the conclusion of the health care procedure, disconnecting the item of medical equipment, removing the sleeve apparatus, and depositing the sleeve apparatus into an appropriate medical waste container.

10. (canceled)

10. The method of claim 9, wherein the thin rubber is blue nitrile rubber, of a 4.0 mm thickness, so as to enable (a) the determination of the location of the wearer's artery and (b the wearer's appropriate artery to be felt underneath the rubber.

11. The method of claim 9, wherein dimensions of length and diameter of the central tubular passage are fabricated so as to accommodate the anatomy, weight, and size of the human wearer for which the tubular sleeve apparatus will be utilized.

12. A method for safeguarding a human wearer from pathogens or harmful organisms that may be conveyed to the surface of the skin of the human wearer when a health care procedure requires placement of a blood pressure cuff, an item of medical equipment or other device against the skin of a limb of the human wearer, the method comprising the steps of:
a) providing a planar, rectangular segment of thin rubber material in a range of dimensions, further having two parallel lateral ends, two parallel longitudinal edges, an interior surface, and an exterior surface;
b) fabricating, on the interior face of the rectangular segment of rubber material, at the first lateral end of the rectangular segment, a strip of material constituting a first means of attachment or bonding with other materials;
c) fabricating on the exterior face of the segment of rubber, and at the second lateral end of the segment, a strip of material constituting a second means of attachment, which corresponds with the first means of attachment;
d) manually rolling the first longitudinal edge of the segment in a curling manner such that the first means of attachment engages the entirety of the length of the second means of attachment at a common seam, thereby forming a tubular sleeve apparatus, having (i) an upper end and a lower end, (ii) a central tubular passage, (iii) a diameter approximately equivalent to the diameter of the subject limb of the human wearer, and (iv) an exterior length sufficient to make contact with the portion of the medical equipment which may impinge upon the skin of the human wearer;
e) placing the sleeve apparatus in direct perimetral contact with the limb about, or upon which the medical equipment is to be placed and ensuring that the interior surface of the material of the tubular sleeve apparatus is placed flush against the skin of the wearer, so as to be absent any folds or gathers;
f) placing the item of medical equipment about, or in abutment with, the newly donned sleeve apparatus; and
g) at the conclusion of the health care procedure, (i) disconnecting the item of medical equipment, (ii) removing the tubular sleeve apparatus from the wearer's limb, and (iii) depositing the tubular sleeve apparatus into an appropriate medical waste container.

13. The method of claim 12, wherein the item of medical equipment comprises a blood pressure cuff and the limb of the wearer comprises either the left or right bicep area.

14. The method of claim 12, wherein the thin rubber material is blue nitrile rubber of 4.0 mm thickness, so as to enable (a) the determination of the location of the wearer's appropriate artery and (b) the wearer's appropriate artery to be felt underneath the rubber.

15. The method of claim 12, wherein the range of dimensions of length and diameter of the central tubular passage are fabricated so as to accommodate the anatomy, weight, and size of the human wearer for which the tubular sleeve apparatus will be utilized.
